(19) United States
(12) Patent Application Publication
(10) Pub. No.: US 2008/0306378 A1
(43) Pub. Date: Dec. 11, 2008

(54) METHOD AND SYSTEM FOR IMAGES REGISTRATION

(76) Inventors: Yves Lucien Trousset, Palaiseau (FR); Jeremie Pescatore, Le Chesnay (FR)

Correspondence Address: General Electric Company;GE Global Patent Operation, PO Box 861, 2 Corporate Drive, Suite 648, Shelton, CT, 06484, US

(21) Appl. No.: 12/055,120

(22) Filed: Mar. 25, 2008

(30) Foreign Application Priority Data
Jun. 5, 2007 (FR) 0704009

(51) Int. Cl. A61B 6/12

(52) U.S. Cl. 600/424

(57) ABSTRACT

A method for registering images acquired by at least one imaging system. The method includes:positioning at least a first sensor (S) interdependent on the imaging system; positioning at least a second sensor (P) so as to make it interdependent on the movements of an organ of a patient of which at least two images are taken; acquiring the positions of said sensors (S, P) during acquisitions of the images; and registering the images according to the positions of the sensors (S, P). A system for registering images acquired by at least one imaging system includes means for acquiring the position of at least a first sensor interdependent on the imaging system and of at least a second sensor interdependent on the movements of an organ of a patient of which two images are taken, the registration being performed according to the positions of the sensors at the moment when the images are acquired.

### CROSS-REFERENCE TO RELATED APPLICATIONS

This application claims the right of priority under 35 USC 119(a)-(d) to the earlier filing date of co-pending French patent application number 07 04009 filed Jun. 5, 2007. The basis for this claim of the right of priority is France's membership in the World Trade Organization.

### BACKGROUND

1. Field of the Invention

Embodiments of the present invention relate to a method for real time registration of images. Advantageously, such embodiments notably find application for registering 3D/3D or 3D/2D images acquired on an angiography system, for registering 3D/3D images acquired on a scanner system (an X-ray, magnetic resonance or ultrasonic scanner), or even for registering 3D/3D or 3D/2D images acquired on a scanner system (X-ray, magnetic resonance, or ultrasonic scanner) and an angiography system.

2. Description of Prior Art

In medical imaging and notably within the scope of interventional radiology it may be useful to acquire images from two different systems.

Indeed, in a preoperative step, one may be led to performing a scan of the organ of the patient on which the operation is intended to be performed. For this purpose, the X scanner (also designated by the acronym CT—for Computed Tomography) may be used, with which a plurality of observed fine sectional views of the region may be obtained, which are then combined in order to form a 3D image.

In this preoperative step, 2D (projective) or 3D (sectional) diagnosis images acquired on an angiography system may also be used. With this step, the practitioner may define the path which the tool should follow in order to reach the targeted region without damaging neighboring organs during its passing.

Next, during the operation, i.e., while introducing the tool and then the operation on the actual targeted region, it is useful for the practitioner to check the position of the tool and how the targeted region is affected. For this purpose, an imaging system is used, the role of which is to help the practitioner to guide the tool. The imaging system used may be the same as the one used in the preoperative step or a different system. For example, the case of an embolization operation on liver tumors, in which the X scanner in the preoperative phase and angiography during the peroperative phase are combined, may be mentioned. Another example is ablation of a cancer tumor of the liver, by radio-frequencies, where the X scanner is used both in the preoperative phase and in the peroperative phase.

During the guiding of the tool, an imaging system may also be coupled to a navigation system. A navigation system has position sensors which are attached to interventional tools or are interdependent on the movements of the organs. The objective of these sensors is to be able to localize in real time the position of the organs and of the tools in 2D or 3D images acquired by the imaging system. However, this device only allows navigation in a same image.

The examined patient or organ may have involuntary movements, such as for example heart beats or breathing. The problem is then posed of registering the images acquired at different instants.

Registering methods are already known to one skilled in the art but they are based on the use of radio-opaque markers stuck on the skin of the patient and visible on the different images, or even anatomic markers, i.e., features of the anatomy of the patient which may be located on the different images. The regsitration method therefore consists of matching visible markers on the images and markers stuck on the skin of the patient or anatomic markers.

In these methods, the movement of the patient is therefore detected by the difference in position of the visible markers on the different images. Registration is therefore only based on the contents of the images.

### SUMMARY OF THE INVENTION

An embodiment of a registration method with which images may be registered automatically by taking into account possible movements of the organ of the patient is disclosed. This method should provide registration of images successively taken over time or even registration of images of a same organ, taken by different imaging systems.

According to an embodiment of the invention, a method for registering images acquired by at least one imaging system is provided, comprising the steps: positioning at least a first sensor interdependent on the imaging system, positioning of at least a second sensor so as to make it interdependent on the movements of an organ of a patient of which at least two images are taken, acquiring positions of said sensors during acquisitions of images, registering the images depending on the positions of the sensors.

Particularly advantageously, said sensors are electromagnetic sensors.

According to a particular embodiment, the second sensor is interdependent on a tool installed in proximity to the organ of the patient so as to adopt the movements of the organ.

According to another feature of the invention, the images are obtained by different imaging systems, and more particularly by an X-ray, magnetic resonance, ultrasonic scanner system, and/or by an angiography system.

Also disclosed is an embodiment of a system for registering images acquired by at least one imaging system. The system may include means for acquiring the position of at least one first sensor interdependent on the imaging system and of at least one second sensor interdependent on the movements of an organ of a patient of which at least two images are taken, and in that the registration is performed depending on the positions of said sensors at the moment when the images are acquired.

Preferably, the registration method described above will be applied by means of a processing device comprising means for applying the steps of the registration method, such as a PC type computer including a memory and a processing unit on which a computer program is executed. This computer program will notably comprise one or more algorithms with which the steps of the method described earlier may be executed. A last object of the invention therefore relates to a computer program, as such, recorded on a medium.

### BRIEF DESCRIPTION OF THE DRAWINGS

Other features and advantages of the invention will become further apparent from the detailed description which follows, from the single appended FIGURE wherein an interventional radiology device is illustrated in which the registration method according to the invention may be applied.

### DETAILED DESCRIPTION

In the field of interventional radiology, different imaging systems may be used, which are well known to one skilled in the art and which will be described briefly.

A widely used imaging system is angiography.

An angiography device typically comprises a table on which lies a patient, an X-ray tube located under the table and a detector interdependent on a gantry, the gantry having the general shape of a C and being mobile along three axes of rotation. Such a device provides acquisition of 2D or 3D images.

In projective 2D imaging, the gantry is held in a determined position and images may be acquired either in a diagnosis context, or in an interventional context, where a catheter is introduced into a blood vessel of the patient.

In diagnosis imaging (radiographic mode), a first image is acquired and then a contrast product is injected in order to take a second image. Subtraction of both images allows the blood vessels to be viewed.

In interventional imaging (fluoroscopic mode), so-called fluoroscopic images are acquired when installing the catheter, in which the location of the latter is viewed.

Moreover, with the angiography device, it is also possible to acquire 3D images. This is then said to be rotational angiography, in which the gantry is displaced according to an amplitude of about 200° and images are acquired according to different inclinations of the gantry. With a tomographic reconstruction algorithm, a 3D image of the vascular system of the patient may be obtained.

Another well-known imaging device is the X-scanner, also called “CT” or “Computed Tomography”.

Generally, this type of device comprises a tunnel equipped with a plurality of detectors, an X-ray tube and a table on which lies the patient and which slides inside the tunnel so as to take several sectional views. With a reconstruction algorithm, a 3D image of the organ of the patient may be reconstructed from different sectional views.

A particular embodiment of the invention will be described wherein both of theses imaging devices are used and it is therefore necessary to register the images from the angiography and the images obtained by the X-scanner.

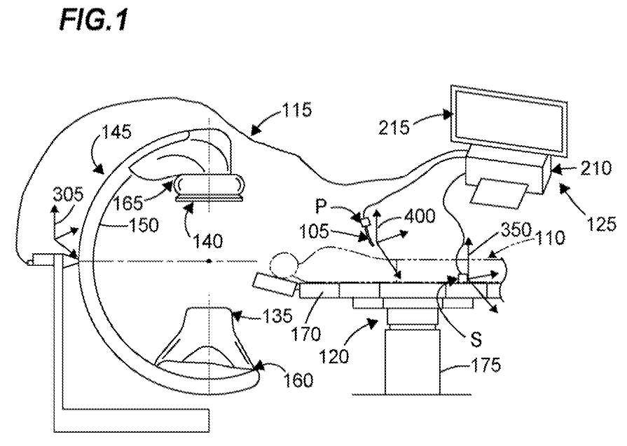

With reference to FIG. 1, a patient 110 lies on a table 120 which is common to both imaging systems used.

The table 120 is typically formed with a platform 170, which is translationally mobile along three axes relatively to a fixed base 175.

The angiography system 115 comprises a gantry 145 formed with a mobile arm having the general shape of a C. The mobile arm may pivot along different axes. One of the ends 165 of the mobile arm 150 supports a detector 140 of the damped X-rays after having crossed a portion of the body of the patient 110 The other end 160 of the mobile arm 150 supports an X-ray source 135.

A X-scanner system, not illustrated in FIG. 1, is installed in the same room so that the table 120 is common to both imaging systems. Translations of the platform 170 of the table allow images to be acquired with either one of the systems, without moving the patient.

The practitioner uses a tool 105 which may for example be a catheter, a guide, a probe, or any other tool suitable for the operation to be performed.

A navigation system 125 is used, which comprises at least sensors, one being associated with the imaging system, the other one with the patient, a workstation 210 is used allowing information sent back by the sensors to be acquired and a viewing screen 215 providing display of the registered images.

At least one electromagnetic sensor S is rigidly fixed to the operating table or to any fixed point. This sensor is common to the X-scanner system and to the angiography system.

At least one second electromagnetic sensor P is intended to adopt the movements of the organ of the patient of which images are desirably taken, so as to take into account movements of the organ and to allow registration by taking into account these movements. For this purpose, several solutions are conceivable for making the sensor P interdependent on the organ.

A first possibility is to place the sensor outside the patient, for example on his/her skin or even on a vertebra.

It is also possible to integrate one or more microsensors into the catheter or the guide; indeed, the catheter, being pressed against the wall of the vessel into which it is introduced, adopts the movement of the latter. In this case, it is considered that the catheter in interdependent on the vessel, without being fixed thereon.

Another possibility of fixing the sensor P to the organ, via a harpoon-shaped needle on which the sensor is fixed and which adheres to the organ.

Finally, a system which may be deployed, may be used by depositing the sensor at the desired location by means of a guide which is then withdrawn.

According to the embodiment illustrated in FIG. 1, the sensor P is fixed to a catheter 105. In order to simplify the diagram, the catheter is illustrated outside the body of the patient, but it is obvious that it is intended to be inserted into a blood vessel of the patient.

The electromagnetic sensors appear in the general form of coils.

Their operation is well known to one skilled in the art and will therefore not be described in detail. The sensors are coupled electromagnetically so as their relative spatial position may be measured.

Each of the sensors is connected to a controller which generally comprises a computer associated with a memory, the computer being able to execute the instructions of programs recorded in the memory for processing the data. The computer may also be controlled by the operator of the imaging system via the workstation 210. The computer outputs the data for displaying the registered images on the viewing screen 215.

In this way, each image taken of the organ may be registered relatively to the other ones by means of the three-dimensional position read for the sensor P by the navigation system.

During the acquisition of the images by both systems, the following spatial information is collected:

the position and the orientation of the sensor S

the position and the orientation of the sensor P

the position of the table during acquisition by the CT system

the position of the table during X-ray acquisition

in the case of 2D acquisition, the position of the gantry.

With the navigation system, it is possible to calculate the spatial relationships between the referentials associated with the table, with the gantry and with the S and P sensors, referenced as 315, 305, 368 and 400, respectively.

Registration is performed simply by adjusting the image according to the three-dimensional displacement of the sensor.

Thus, for example, the matrix with which it is possible to pass from an 3D angiography acquisition system to an X-scanner (CT) acquisition system will be given by:

M_{G}^{CT}=M_{S}^{P}^{_{CT}}M_{T}_{_{3Dacq}}^{G}M_{P}_{_{3Dacq}}^{S}M_{CT}^{T}^{_{CT}} (Eq. 1)

wherein the index G refers to the referential of the gantry, the index T refers to the referential of the table, and the notation of the type M_{S}^{P}^{_{CT}} illustrates the matrix with which it is possible to pass from the referenctial of the sensor S to the referential of the sensor P during the X-scanner acquisition.

Indeed, during the CT acquisition, one has:

M_{CT}^{P}^{_{CT}}=M_{S}^{P}^{_{CT}}M_{T}_{_{CT}}^{S}M_{CT}^{T}^{_{CT}} (Eq. 2)

and during the 3D angiographic acquisition, one has:

M_{G}^{P}^{_{3Dacq}}=M_{S}^{P}^{_{3Dacq}}M_{T}_{_{3Dacq}}^{S}M_{G}^{T}^{_{3Dacq}} (Eq. 3)

Now, we assume that:

M_{T}_{_{3Dacq}}^{S}=M_{T}_{_{CT}}^{S} (Eq 4)

and:

M_{CT}^{G}=M_{P}_{_{3Dacq}}^{G}M_{CT}^{P}^{_{CT}} (Eq. 5)

The invention finds application in many cases, and may be used in an X-scanner/angiography hybrid system, just as in a system only based on the X-scanner or a system only based on angiography.

### First Application Example: “CT/2D Registration” (Images Obtained by X-Scanner and by Angiography)

A first application example is a hybrid system using the X-scanner (CT) and angiography. Such a hybrid system may be used in operations which require catheterization, e.g. for embolization by catheter.

Embolization is a technique with which one or more abnormal blood vessels or responsible for bleedings may be occluded, by means of a material, the nature of which depends on the type of occlusion and on the diamater of the vessels to be treated.

Before the operation, a catheter is inserted into the blood system of the patient so that the end of the catheter is found in proximity to the vessel to be closed. An electromagnetic sensor P is integrated to the catheter and is therefore found positioned against the wall of the vessel to be treated, of which it adopts the movements. A 3D image is then acquired of the region to be treated by the X-scanner. During this acquisition, the navigation system records the position of the sensor P relatively to the sensor S.

One next proceeds with embolization by inserting the adequate material into the catheter. To track the course of this operation, 2D fluoroscopic image acquisitions are performed, during which the navigation system also records the position of the sensor P relatively to the sensor S.

The registration method allows the preoperative image obtained by the X-scanner to be automatically registered with the 2D fluoroscopic images taken during embolization.

### Second Example of Application: “CT/CT Registration” (Images Obtained by Means of an X-Scanner)

Another example of application of the registration method according to the invention is the ablation of a liver tumor by radiofrequencies.

Before proceeding with the actual operation, the patient lying on the table, the table is equipped with an electromagnetic sensor S and the liver of the patient with an electromagnetic sensor P. Acquisition of a 3D image from the relevant region by means of the X-scanner is then performed. During this acquisition, the navigation system records the position of the sensor P relatively to the sensor S.

Next, one proceeds with ablation of the tumor, which consists of introducing a radiofrequency probe into the tumor by guiding it with an imaging technique such as the X-scanner, and then, once the probe is positioned in the tumor, of having a radiofrequency current pass into the probe in order to heat and remove the tumoral tissue located near the end of the probe. In the peroperative phase, the same imaging system provides acquisition of fluoroscopic sectional views of the region of interest. During these acquisitions, the navigation system also records the position of the sensor P relatively to the sensor S.

With the registration method, it is possible to automatically register the preoperative 3D image on the scanning sectional views (scanner fluoroscopic mode or CT mode, a few sectional views) taking into account movements—notably breathing movements—of the patient.

Further, the advantage of this method is that the virtual tool may be viewed in the 3D image, so that the target to be burnt may be seen better than in the fluoroscopic images in which the metal needle and electrode generate substantial artifacts.

The use of this method therefore makes ablation of liver tumors faster and safer.

### Third Example of Application: “3D/2D Registration” (Images Obtained by Angiography)

In the case of an angiography system, with the invention, a 3D acquisition may be merged with a fluoroscopic acquisition while taking into account movements. Thus, registration of 3D images of the heart and of 2D images within the framework of the ablation of an atrial fibrilation may be used. Atrial fibrilation is defined as the anarchical contraction of the atria, which causes fast and irregular contraction of the ventricles located just below them. It may notably be treated by inserting, via a catheter passing through the atrium, a radiofrequency probe at the pulmonary veins, in order to perform a “shot” thereon, which will have the purpose of damaging an area placed under the probe. A succession of lesions will have the purpose of isolating the foci responsible for occurrence of atrial fibrilation.

As this was seen, with the invention, registration of images obtained in different contexts may be obtained, such as:

time registration of several 3D images obtained by angiography (“3D/3D registration”)

time registration of several CT images (“CT/CT registration”)

time registration of several 2D images (“2D/2D registration”)

time registration of several 3D and 2D images (“3D/2D registration”)

registration of images by X-scanning with 2D images (“CT/2D registration”).

Of course the invention is not limited to the imaging systems mentioned earlier, but it may also be applied to images obtained by means of a magnetic resonance scanner (MR) or even by an ultrasound scanner.

Moreover, the registration between the X-scanner and angiography systems does not require the use of any marker on the patient as the movement of the patient or of the organ are detected by means of electromagnetic sensors.

In this respect, it will be noted that any sensor capable of taking into account movements of the organ may be used. For example, outside the body of the patient, optical sensors may also be used.

Registration therefore does not use the contents of the image, unlike the methods from the prior art.

Finally, it is obvious that the examples which have just been given are only particular illustrations, by no means limiting as to the fields of application of the invention. The field of application is notably not limited to medical imaging but to any technical field which involves registration of images. Moreover, other types of three-dimensional sensors may be used.

1.-7. (canceled)

8. A method for registering images acquired by at least one imaging system, the method comprising:
positioning at least one first sensor (S) interdependent on the imaging system;
positioning at least one second sensor (P) so as to make it interdependent on the movements of an organ of a patient of which at least two images are taken;
acquiring the positions of said sensors (S, P) during acquisitions of images;
registering the images according to the positions of the sensors (S, P).

9. The method according to claim 1, wherein said sensors (S, P) are electromagnetic sensors.

10. The method according to claim 2, wherein the second sensor (P) is interdependent on a tool installed in proximity to the organ of the patient so as to adopt the movements of the organ.

11. The method according to claim 1, wherein the images are obtained by different imaging systems.

12. The method according to claim 4, wherein the images are obtained by an X-ray system, a magnetic resonance system, ultrasound scanner system and/or an angiography system.

13. A system for registering images acquired by at least one imaging system, the system comprising:
means for acquiring the position of at least a first sensor (S) interdependent on the imaging system and of at least a second sensor (P) interdependent on the movements of an organ of a patient of which at least two images are taken, and in that registration is performed according to the positions of said sensors (S, P) at the moment when the images are acquired.
